(19) United States
(12) Patent Application Publication
(10) Pub. No.: US 2023/0320856 A1
(43) Pub. Date: Oct. 12, 2023

(54) ANNULAR REPAIR OF THE TRICUSPID VALVE AND MITRAL VALVE

(71) Applicant: Ventrimend, Inc, Lake Forest, CA (US)

(72) Inventors: David Zarbatany, Lake Forest, CA (US); Ricardo Roman, Lake Forest, CA (US); Jesus Flores, Lake Forest, CA (US)

(73) Assignee: Ventrimend, Inc, Lake Forest, CA (US)

(21) Appl. No.: 18/106,444

(22) Filed: Feb. 6, 2023

Related U.S. Application Data
(60) Provisional application No. 63/306,966, filed on Feb. 4, 2022.

(51) Int. Cl. A61F 2/24; A61B 17/34

(52) U.S. Cl. CPC A61F 2/2466; A61F 2/2454; A61B 17/3468; A61F 2220/0041

(57) ABSTRACT

The present invention describes systems and methods for treating tricuspid valve regurgitation and mitral valve regurgitation. The treatment includes a systems and method of modifying the tricuspid or mitral valve by attaching a device in two locations in the valve annulus and pulling them toward each other to stop valve regurgitation by modifying the leaflets of the valve.

### CROSS-REFERENCE TO RELATED APPLICATIONS

This application claims priority to U.S. Provisional Patent Application No. 63/306,966, filed Feb. 4, 2022, the entire disclosure of which is incorporated by reference herein.

### FIELD

The present invention relates generally to the field of surgery, and more specifically, to repair of a tricuspid valve.

### BACKGROUND

The heart has four valves that keep blood flowing in the correct direction. The valves include the mitral valve, tricuspid valve, pulmonary valve and aortic valve. Each valve has flaps (leaflets or cusps) that open and close once during each heartbeat. Sometimes, the valves don't open or close properly, disrupting the blood flow through your heart to your body.

The tricuspid valve lies between the right atrium and the right ventricle and usually has three leaflets, named the anterior, posterior, and septal leaflets. The function of the tricuspid valve is to prevent back flow (regurgitation) of blood from the right ventricle into the right atrium during right ventricular contraction.

Tricuspid valve regurgitation is a condition in which the valve between the two right heart chambers (right ventricle and right atrium) does not close completely when the right ventricle contracts. The malfunctioning valve allows blood to flow backwards from the right ventricle to the right atrium, which increases the volume and pressure of the blood, both in the right atrium and the right ventricle, which may increase central venous volume and pressure if the backward flow is sufficiently severe.

The mitral valve lies between the left atrium and the left ventricle. Normally, the mitral valve prevents blood flowing back into the left atrium from the left ventricle. When the mitral valve becomes leaky, it's called mitral valve regurgitation. Mitral valve regurgitation is a condition in which the mitral valve leaflets do not close tightly, allowing blood to flow backward through the mitral valve each time the left ventricle contracts. A leaking mitral valve allows blood to flow in two directions during the contraction. Some blood flows from the ventricle through the aortic valve and some blood flows back into the atrium. Leakage can increase blood volume and pressure in the left atrium. The increased pressure can increase pressure in the veins leading from the lungs to the heart (pulmonary veins). If regurgitation is severe, increased pressure may result in congestion (or fluid build-up) in the lungs. Because of this, the heart has to work harder than it should to get blood out to the body.

Accordingly, there is a need for systems and methods that provide solutions to repair tricuspid valve and mitral valve regurgitation. The present invention is directed toward systems and methods for treating these problems.

### SUMMARY

The present invention describes systems and methods for treating tricuspid valve regurgitation and mitral valve regurgitation. The tricuspid valve treatment includes a systems and method of modifying the tricuspid valve by attaching a device between a pair of leaflets and pulling them toward each other to stop tricuspid valve regurgitation by modifying the tricuspid valve to operate as a bicuspid valve. The mitral valve treatment includes a systems and method of modifying the mitral valve by attaching a device proximate the posterior leaflet to reshape the mitral valve to operate as a normal mitral valve and to stop mitral valve regurgitation.

### BRIEF DESCRIPTION OF THE DRAWINGS

FIG. 1 is a sectional view of the heart anatomy.

FIG. 2A is a view of a normal tricuspid valve with three leaflets: the anterior leaflet, posterior leaflet, and septal leaflet.

FIG. 2B is a view of a tricuspid valve with the leaflets not functioning properly leading to back flow of blood is also known as tricuspid valve regurgitation.

FIG. 3A is a view of a normal mitral valve with two leaflets: the anterior leaflet and posterior leaflet.

FIG. 3B is a view of a mitral valve with the leaflets not functioning properly leading to back flow of blood is also known as mitral valve regurgitation.

FIG. 4 shows a delivery system advanced to the abnormal or damaged tricuspid valve.

FIGS. 5-6 show an overview of one embodiment of treatment for tricuspid valve regurgitation.

FIG. 7A is a view and 7B is a sectional view at A-A showing the modified tricuspid valve.

FIGS. 8A-10B show an overview of another embodiment of treatment for tricuspid valve regurgitation with the addition of pledgets.

FIG. 11 shows a delivery system advanced to the abnormal or damaged mitral valve.

FIGS. 12-13 show an overview of one embodiment of treatment for mitral valve regurgitation.

FIG. 14A is a view and 14B is a sectional view at E-E showing the modified mitral valve.

FIGS. 15A-17B show an overview of another embodiment of treatment for tricuspid valve regurgitation with the addition of pledgets.

FIG. 18 shows delivery of a delivery system having a guide catheter that enters in the femoral vein and is advanced through the vascular system to the heart and into the right atrium.

FIG. 19 shows the integrated therapy catheter advanced to a first location, junction A, of the tricuspid valve and puncture a first hole in the tricuspid valve annulus.

FIG. 20 shows the integrated therapy catheter advanced to the opposite side of the tricuspid valve and puncture a second hole in the tricuspid valve annulus at a second location, junction B.

FIG. 21 shows the proximal end of the first and second tethers inserted into a knot replacement tool.

FIG. 22 shows the knot replacement tool advancing over the first and second tethers toward the tricuspid valve.

FIG. 23 shows the final position between junction A and junction B and the knot replacement tool locking the first and second tethers together.

FIG. 24 shows the proximal end of the first and second tethers inserted into a tether cutter.

FIG. 25A is a top view and 25B is a sectional view at I-I showing the knot replacement tool in the locked position with the first and second tethers.

FIG. 26 shows another embodiment of treatment with the proximal end of the first and second tethers inserted through first and second pledgets, the first and second pledgets are then advanced along the first and second tethers toward junctions A, B.

FIG. 27 shows the first pledget positioned on the tricuspid valve annulus tissue at junction A, opposite the first anchor, and the second pledget positioned on the annulus tissue at junction B, opposite the second anchor.

FIG. 28 shows the final position between junction A and junction B and the knot replacement tool locking the first and second tethers together.

FIG. 29 shows a tether cutter advancing toward junctions A, B to cut the first and second tethers near the knot replacement tool.

FIG. 30A is a top view and 30B is a sectional view at J-J showing the knot replacement tool in the locked position with the first and second tethers.

FIG. 31 shows delivery of a delivery system having a guide catheter that enters in the femoral vein and is advanced through the vascular system to the heart and into the right atrium.

FIG. 32 shows the integrated therapy catheter advanced to a first location, location C, of the tricuspid valve and puncture a first hole in the tricuspid valve annulus.

FIG. 33 shows the integrated therapy catheter advanced to the opposite side of the tricuspid valve and puncture a second hole in the tricuspid valve annulus at a second location, location D.

FIG. 34 shows the proximal end of the first and second tethers inserted into a knot replacement tool.

FIG. 35 shows the knot replacement tool advancing over the first and second tethers toward the tricuspid valve.

FIG. 36 shows the final position between location C and location D and the knot replacement tool locking the first and second tethers together.

FIG. 37 shows the proximal end of the first and second tethers inserted into a tether cutter.

FIG. 38A is a top view and 38B is a sectional view at K-K showing the knot replacement tool in the locked position with the first and second tethers.

FIG. 39 shows another embodiment of treatment with the proximal end of the first and second tethers inserted through first and second pledgets, the first and second pledgets are then advanced along the first and second tethers toward locations C and D.

FIG. 40 shows the first pledget positioned on the tricuspid valve annulus tissue at location C, opposite the first anchor, and the second pledget positioned on the annulus tissue at location D, opposite the second anchor.

FIG. 41 shows the final position between junction A and junction B and the knot replacement tool locking the first and second tethers together.

FIG. 42 shows a tether cutter advancing toward junctions A, B to cut the first and second tethers near the knot replacement tool.

FIG. 43A is a top view and 43B is a sectional view at L-L showing the knot replacement tool in the locked position with the first and second tethers.

FIGS. 44-50 show a perspective view and side views of an integrated therapy catheter configured to: engage a tricuspid valve at the junction A or junction B, puncture the tricuspid valve, and deliver first and second anchors into the tricuspid valve annulus at junction A or junction B.

FIGS. 51-54 are views showing one embodiment of a knot replacement tool.

FIG. 55 is a sectional view showing the delivery shaft coupled to the shaft coupler.

FIG. 56 is a sectional view showing the delivery shaft disengaged from the shaft coupler.

FIGS. 57-61 are views showing one embodiment of a cutter.

FIGS. 62-66 are views showing the knot replacement tool.

### DETAILED DESCRIPTION

The tricuspid valve has three leaflets, named the anterior, posterior, and septal leaflets. The mitral valve has two leaflets, named the anterior and posterior leaflets. Embodiments of the present invention describe devices, systems and methods for treating tricuspid valve regurgitation or mitral valve regurgitation by modifying the leaflets of abnormal or damaged valves by attaching multiple anchors at multiple locations in the valve annulus and then pulling the anchors toward each other to modify the leaflets to stop valve regurgitation.

One embodiment of the present invention describes systems and methods for treating tricuspid valve regurgitation by modifying the tricuspid valve to operate as a bicuspid valve by attaching first and second anchors at first and second locations in the valve annulus, then pull the first and second anchors toward each other to create the bicuspid valve using the anterior and septal leaflets to stop tricuspid valve regurgitation. In the embodiments shown below, the first location is junction A of the anterior/posterior leaflets and the second location is junction B of the septal/posterior leaflets.

A therapy catheter initially engages the tricuspid valve annulus above the first junction of the anterior/posterior leaflets and the tricuspid valve annulus above the second junction above the septal/posterior leaflets, then a therapy catheter needle is used to puncture through the first and second junctions. The therapy catheter is used to deliver first and second anchors with first and second tethers. The first and second anchors are then attached and pulled toward each other. The first and second anchors are then fixed in the desired position.

The steps include a therapy catheter directed to a tricuspid valve through a double steerable sheath system. The outer sheath/transseptal sheath is placed in the femoral vein and is advanced through the vascular system to the heart and into the right atrium. The inner steerable/guiding catheter is placed through the outer sheath and deflected to set a trajectory to the target first and second junctions A, B.

The therapy catheter, internal to the inner steerable, includes a cannulated needle used to puncture the tricuspid valve at the desired first and second junctions A, B.

Another embodiment of the present invention describes systems and methods for treating mitral valve regurgitation by modifying the posterior leaflet by attaching first and second anchors at the first and second locations above the posterior leaflet in the mitral valve annulus, then pull the first and second anchors toward each other to modify the shape of the posterior leaflet to stop mitral valve regurgitation. In the embodiments shown below, the first location is location C and the second location is location D above the posterior leaflet.

A therapy catheter initially engages the mitral valve annulus above the posterior leaflet at the first and second locations, then a therapy catheter needle is used to puncture through the leaflet at the first and second locations. The therapy catheter is used to deliver first and second anchors with first and second tethers. The first and second anchors are then attached and pulled toward each other. The first and second anchors are then fixed in the desired position.

The steps include a therapy catheter directed to a mitral valve through a double steerable sheath system. The outer sheath/transseptal sheath is placed in the femoral vein and is advanced through the vascular system to the heart and into the left atrium. The inner steerable/guiding catheter is placed through the outer sheath and deflected to set a trajectory to the target first and second location of the mitral valve.

The therapy catheter, internal to the inner steerable, includes a cannulated needle used to puncture the valve at the desired first and second locations.

First and second anchors with tethers are delivered through the cannulated needle and the puncture site. The first and second anchors expand into the valve annulus or adjacent wall. The first and second tethers are exposed as the catheter is removed.

The proximal end of the first and second tethers are inserted into the knot replacement tool. The knot replacement tool uses the first and second tethers as a rail to guide it to the tricuspid valve.

The knot replacement is positioned at the leaflets, the first and second tethers are tensioned independently, and the knot replacement is engaged and released, leaving the tethers exposed at the groin.

The Tether Cutting Catheter follows the first and second tethers to the knot replacement tool and cuts the first and second tethers.

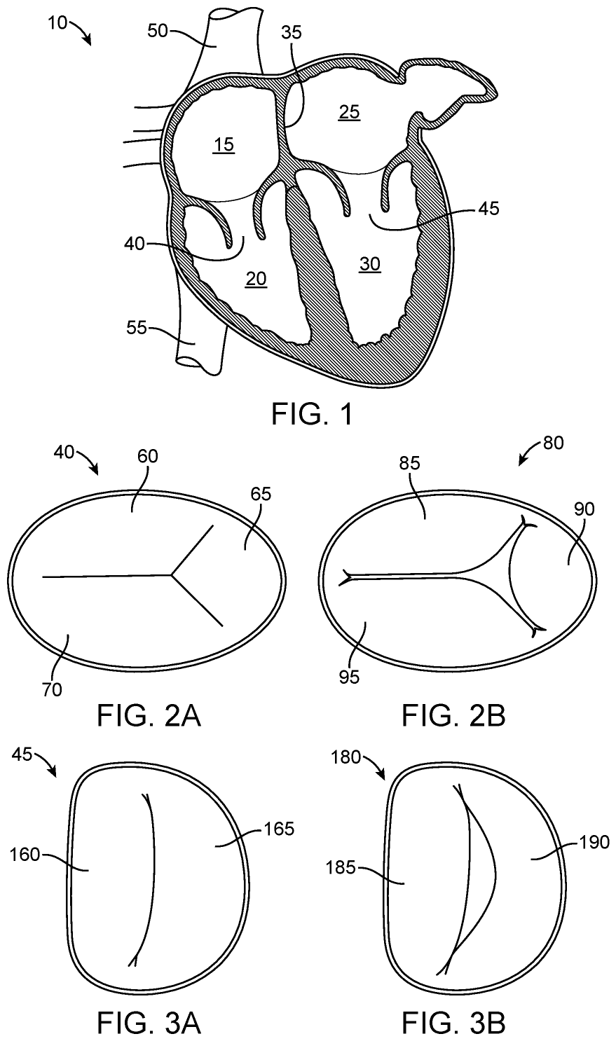

FIG. 1 is a sectional view of a normal heart anatomy 10. The heart includes four chambers, including a right atrium 15, a right ventricle 20, a left atrium 25 and a left ventricle 30. The right atrium 15 and left atrium 25 are separated by the atrial septum 35. A tricuspid valve 40 allows one way blood to flow from the right atrium 15 into the right ventricle 20. A mitral valve 45 allows one way blood to flow from the left atrium 25 into the left vertical 30.

Blood enters the right atrium 15 from the superior vena cava 50 and the inferior vena cava 55 blood vessels. The blood flows into the right atrium 15, through the tricuspid valve 40 into the right ventricle 20. Blood then flows from the right ventricle 20 into the pulmonary arteries to the lungs. Once through the lungs, the blood flows through the pulmonary veins back to the heart and into the left atrium 25. The blood from the left atrium 25 flows through the mitral valve 45 into the left ventricle 30 and out of the heart through the aortic valve to the ascending aorta.

FIG. 2A is a view of a normal tricuspid valve 40 having three leaflets: the anterior leaflet (AL) 60, posterior leaflet (PL) 65, and septal leaflet (SL) 70. The tricuspid valve functions as a one-way valve that closes during ventricular systole to prevent regurgitation of blood from the right ventricle back into the right atrium. It opens during ventricular diastole, allowing blood to flow from the right atrium into the right ventricle.

FIG. 2B is a view of a tricuspid valve 80 with three leaflets: the anterior leaflet (AL) 85, posterior leaflet (PL) 90, and septal leaflet (SL) 95, that are not functioning properly, leading to back flow of blood, known as tricuspid valve regurgitation. Tricuspid valve regurgitation can result in increased ventricular preload because the blood refluxed back into the atrium is added to the volume of blood that must be pumped back into the ventricle during the next cycle of ventricular diastole. Increased right ventricular preload over a prolonged period of time may lead to right ventricular enlargement which can progress to right heart failure.

FIG. 3A is a view of a normal mitral valve 45 having two leaflets: the anterior leaflet (AL) 160 and posterior leaflet (PL) 165. The mitral valve functions as a one-way valve, blood flows through an open mitral valve during diastole with contraction of the left atrium, and the mitral valve closes during systole with contraction of the left ventricle. The valve opens and closes because of pressure differences, opening when there is greater pressure in the left atrium than ventricle and closing when there is greater pressure in the left ventricle than atrium.

FIG. 3B is a view of a leaking mitral valve 180 with the leaflets not functioning properly, anterior leaflet (AL) 185 and posterior leaflet (PL) 190, leading to blood flowing back into the left atrium from the left ventricle, known as mitral valve regurgitation. Mitral valve regurgitation is a condition in which the mitral valve leaflets do not close tightly, allowing blood to flow backward through the mitral valve each time the left ventricle contracts. A leaking mitral valve allows blood to flow in two directions during the contraction. Some blood flows from the ventricle through the aortic valve and some blood flows back into the atrium. Leakage can increase blood volume and pressure in the left atrium. The increased pressure can increase pressure in the veins leading from the lungs to the heart (pulmonary veins). If regurgitation is severe, increased pressure may result in congestion (or fluid build-up) in the lungs. Because of this, the heart has to work harder than it should to get blood out to the body.

Tricuspid Valve Repair Overview

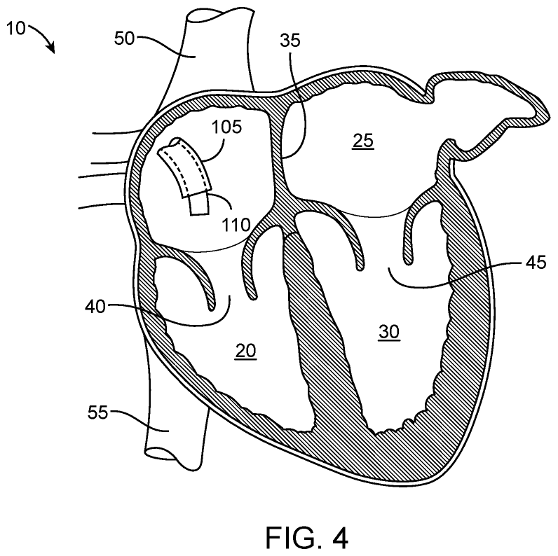

FIG. 4 shows a delivery system 100 having a guide catheter 105 that enters in the femoral vein and is advanced through the vascular system to the heart 10 and into the right atrium 15. A second steerable sheath 110, such as an inner steerable/guide, is inserted through the guide catheter 105 and exits the distal end and is advanced to the abnormal or damaged tricuspid valve 80.

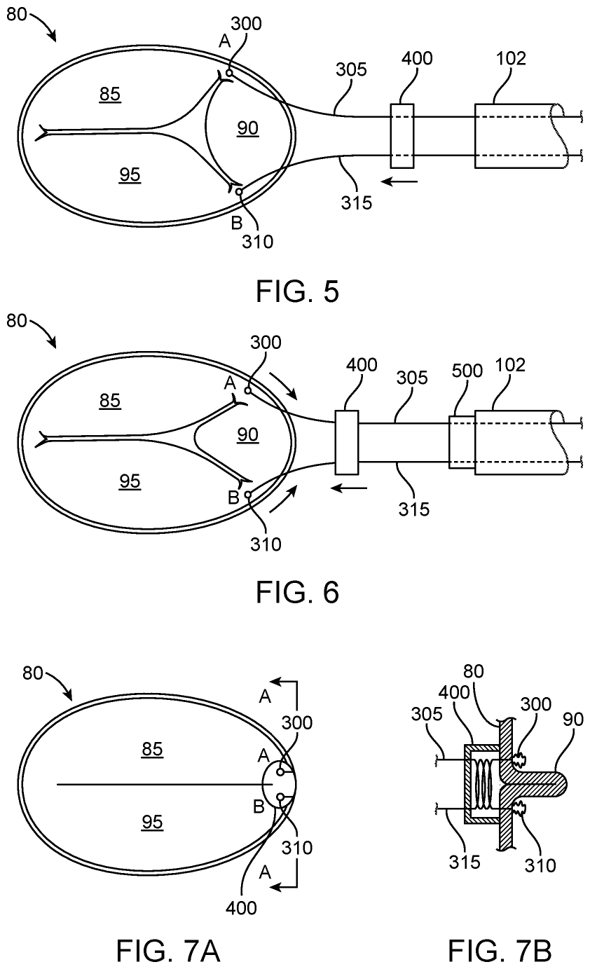

FIGS. 5-7B show an overview of treatment for tricuspid valve regurgitation using the present invention. The treatment includes systems and methods of modifying the abnormal or damaged tricuspid valve 80 to operate as a bicuspid valve to stop the tricuspid valve regurgitation. This is done by attaching first and second anchors in the annulus above the damaged leaflets and pulling the annulus above the leaflets toward each other to create the bicuspid valve using the anterior leaflet and septal leaflet. It may take multiple anchors that are pulled together to completely isolate the posterior leaflet.

FIG. 5 shows attachment of first anchor 300 and first tether 305, and second anchor 310 and second tether 315. The first anchor 300 is attached into the tricuspid valve annulus at junction A above the anterior leaflet 85 and posterior leaflet 90. The second anchor 310 is attached into the tricuspid valve annulus at junction B above the posterior leaflet 90 and septal leaflet 95. The proximal ends of the first and second tethers 305, 315 are inserted into a knot replacement tool 400. The knot replacement tool 400 uses the first and second tethers 305, 315 as a rail to guide it to the tricuspid valve 80.

FIG. 6 shows the knot replacement tool 400 advancing along the first and second tethers 305, 315 and tensioning the first and second tethers 305, 315 so that the anchors 300, 310 at junction A and junction B move toward each other and the anterior leaflet 85 and the septal leaflet 95 are brought together.

Once the anterior leaflet 85 and the septal leaflet 95 are brought together, the knot replacement tool 400 ties the first and second tethers 305, 315, locking the anterior leaflet 85 and septal leaflet 95 in place.

A cutter 500 follows the first and second tethers 305, 315 to the knot replacement tool 400. The cutter 500 cuts the proximal end of the first and second tethers 505, 515 and then is withdrawn from the guide catheter 100.

FIG. 7A shows the knot replacement tool 400 in the locked position with the first and second tethers 305, 315, locking the anterior leaflet 85 and septal leaflet 95 in place. The creates the modified tricuspid valve 80 with the anterior leaflet 85 and the septal leaflet 95 functioning as a bicuspid valve.

FIG. 7B is a sectional view at A-A showing the modified tricuspid valve 80.

Tricuspid Valve—Optional Pledgets

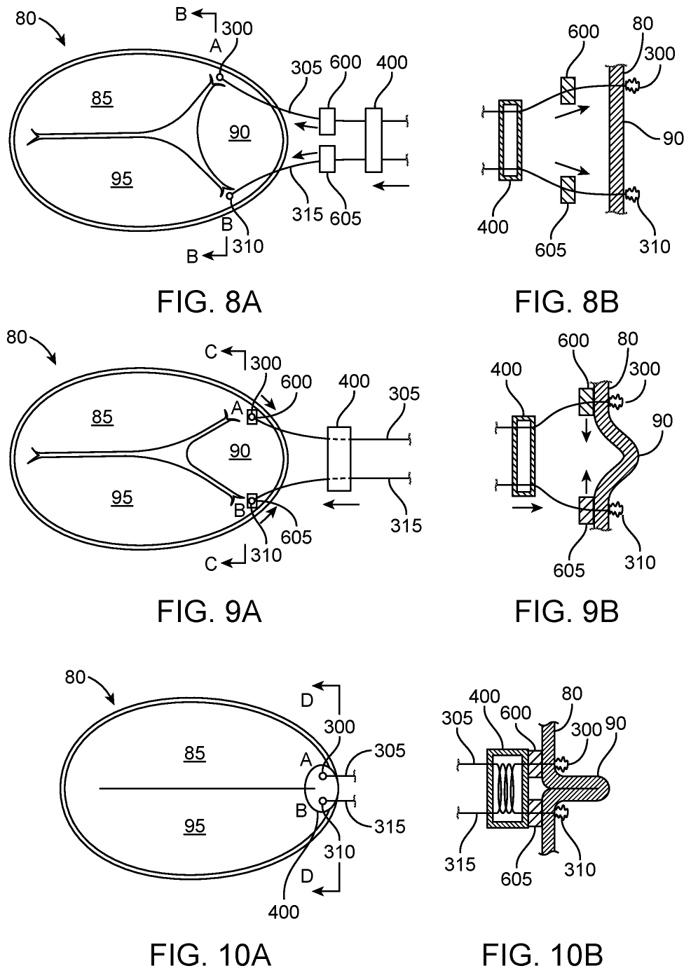

FIGS. 8A-10B are similar to FIGS. 5-7B with the addition of pledgets in the treatment for tricuspid valve regurgitation. In the embodiments shown, pledgets may be positioned against the annulus tissue to prevent tearing of the annulus tissue when the knot replacement tool 400 is pulling the first anchor 300 and the second anchor 310 towards each other and tying the first and second tethers 305, 315 together. The pledgets 600, 605 are design to be delivered on the first and second tethers 305, 315 and contact the annulus tissue of the tricuspid valve at junction A and junction B.

The pledgets may be made from biocompatible flexible materials such as foam, felt or fabric. Pledgets may also be made from non-resorbable polyurethane, polyamide, polyethylene, polypropylene, polyethylene terephthalate, polytetrafluoroethylene (PTFE or Teflon®), or various absorbable polymers, such as polyglycolic acid or even pieces of autologous tissue. Pledgets may be formed in different shapes, such as circular or rectangular.

The treatment for tricuspid valve regurgitation includes systems and methods of modifying the abnormal or damaged tricuspid valve 80 to operate as a bicuspid valve to stop the tricuspid valve regurgitation. This is done by attaching first and second anchors 300, 310 in the annulus above the damaged leaflets and pulling the annulus above the leaflets toward each other. The pledgets 600, 605 are used to prevent tearing of the annulus tissue when tension is applied to move the first and second anchors 300, 310 toward each other to create the bicuspid valve using the anterior leaflet 85 and septal leaflet 95. It may take multiple anchors that are pulled together to completely isolate the posterior leaflet 90.

FIG. 8A is a top view and 8B is a sectional view at B-B showing the first anchor 300 attached to the tricuspid valve annulus at junction A above the anterior leaflet 85 and posterior leaflet 90. The second anchor 310 is attached into the tricuspid valve annulus at junction B above the posterior leaflet 90 and septal leaflet 95. First and second pledgets 600, 605 are advanced along the first and second tethers 305, 315 to junction A and junction B. Following the pledgets 600, the knot replacement tool 400 is advanced along the first and second tethers 305, 315.

FIG. 9A is a top view and 9B is a sectional view at C-C showing the first pledget 600 positioned at junction A and the second pledget 605 positioned at junction B. The knot replacement tool 400 is then advanced along the first and second tethers 305, 315. While advancing, the knot replacement tool 400 applies tension to the first and second tethers 305, 315 so that the first anchor 300 at junction A moves toward the second anchor 310 at junction B and the anterior leaflet 85 and the septal leaflet 95 are brought closer together.

Once the anterior leaflet 85 and the septal leaflet 95 are brought together, the knot replacement tool 400 ties the first and second tethers 305, 315, locking the anterior leaflet 85 and septal leaflet 95 in place.

A cutter 500 follows the first and second tethers 305, 315 to the knot replacement tool 400. The cutter 500 cuts the proximal end of the first and second tethers 505, 515 and then is withdrawn from the guide catheter 100.

FIG. 10A is a top view and 10B is a sectional view at D-D showing the knot replacement tool 400 in the locked position with the first and second pledgets 600, 605 positioned against the annulus tissue on the first and second tethers 305, 315, locking the anterior leaflet 85 and septal leaflet 95 in place, creating the modified tricuspid valve 80, with the anterior leaflet 85 and the septal leaflet 95 functioning as a bicuspid valve.

Mitral Valve Repair Overview

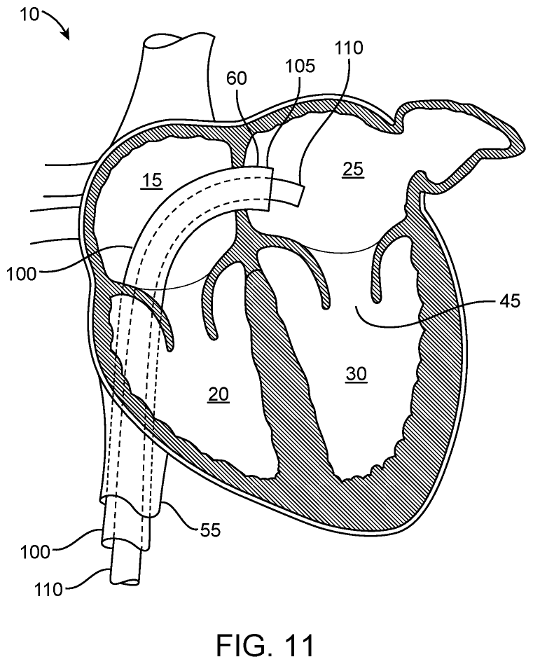

FIG. 11 shows delivery of a delivery system 100, such as an outer steerable/transseptal sheath, that is advanced through the vascular system and into the heart 10 to the left atrium 25. The delivery system 100 enters in the femoral vein and is advanced through the vascular system to the heart and into the right atrium 15. The delivery system 100 then goes through the atrial septum 35 to left atrium 25. This can be done by puncturing a hole 60 in the wall of the atrial septum 35 between the right atrium 15 and the left atrium 25, or through a hole 60 of a patent foramen ovale or atrial septal defect, if present. A second steerable sheath 110, such as an inner steerable/guide, is inserted through the delivery system 100 and exits the distal end 105 in the left atrium 25. The second steerable sheath 110 then sets the trajectory toward the mitral valve 45. Once in the left atrium 25, the distal end 115 of the second steerable sheath 110 is advanced to the mitral valve 45.

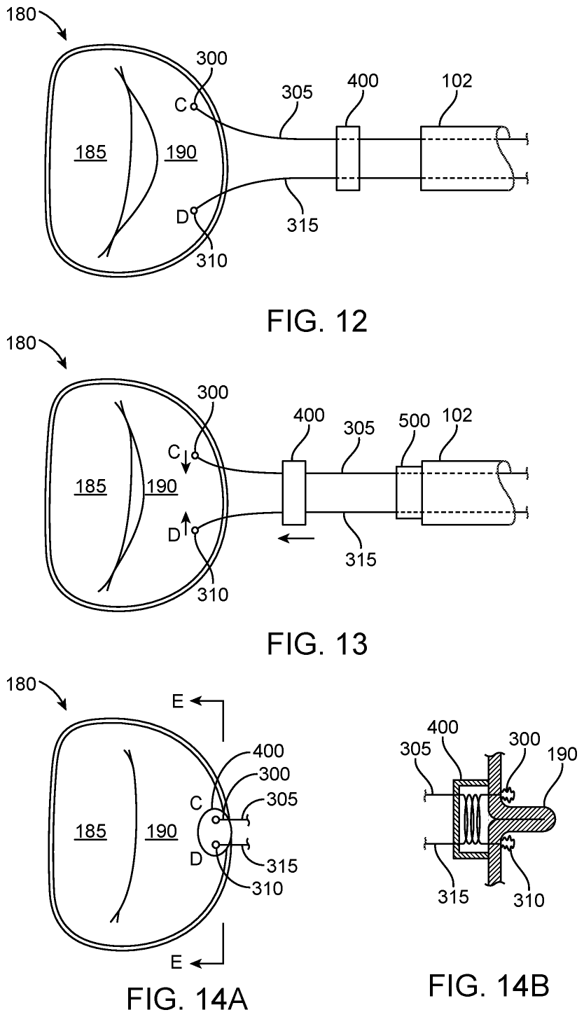

FIGS. 12-14B show an overview of treatment for mitral valve regurgitation using the present invention. The treatment includes systems and methods of modifying the abnormal or damaged mitral valve 180 to stop the mitral valve regurgitation. This is done by attaching first and second anchors 300, 310 in the annulus of the posterior leaflet 190 and pulling the first and second anchors toward each other to reshape the posterior leaflet 190 so that it will function properly.

FIG. 12 shows attachment of the first anchor 300 and first tether 305, and the second anchor 310 and second tether 315. The first and second anchors 300, 310 are attached to the mitral valve annulus at locations C and D above the posterior leaflet 190. The proximal ends of the first and second tethers 305, 315 are inserted into a knot replacement tool 400. The knot replacement tool 400 uses the first and second tethers 305, 315 as a rail to guide it to the mitral valve 180.

FIG. 13 shows the knot replacement tool 400 advancing along the first and second tethers 305, 315 and tensioning the first and second tethers 305, 315 so that anchor 300 at location C and the anchor 310 at location D move toward each other, reshaping the posterior leaflet 190. Once the posterior leaflet 190 is reshaped the desired amount, the knot replacement tool 400 ties the first and second tethers 305, 315, locking the modified posterior leaflet 190 in place.

A cutter 500 follows the first and second tethers 305, 315 to the knot replacement tool 400. The cutter 500 then cuts the proximal end of the first and second tethers 505, 515 and is withdrawn from the guide catheter 100.

FIG. 14A shows the knot replacement tool 400 in the locked position with the first and second tethers 305, 315, locking the modified posterior leaflet 190 in place. This creates the modified tricuspid valve 180 with the anterior leaflet 185 and the posterior leaflet 190 functioning similar to a normal mitral valve 45.

FIG. 14B is a sectional view at E-E showing the modified mitral valve 180.

Mitral Valve Optional Pledgets

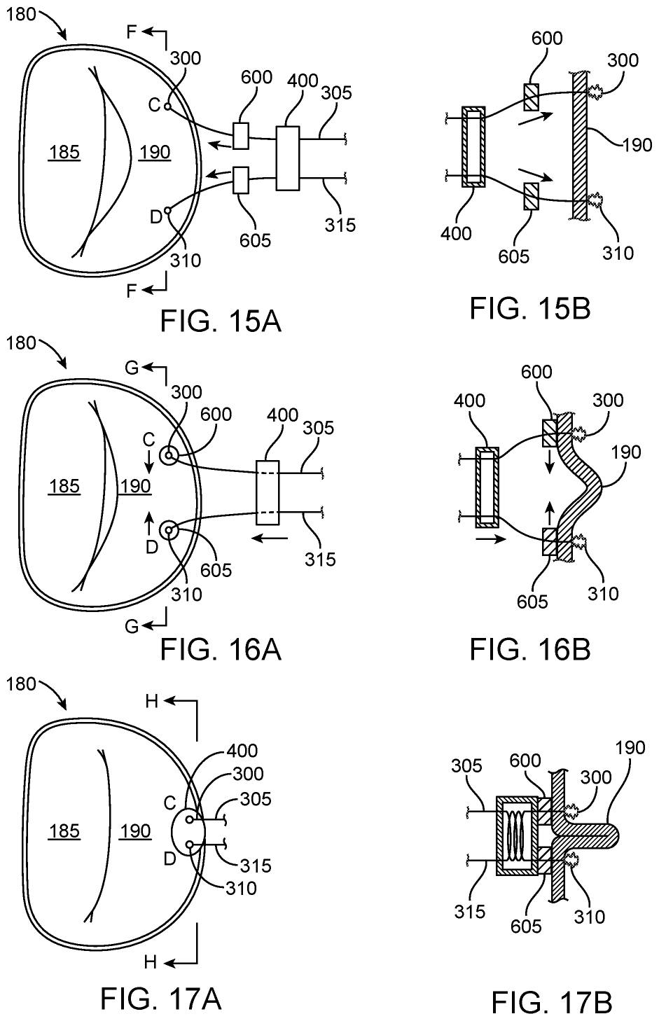

FIGS. 15A-17B are similar to FIGS. 12-14B with the addition of pledgets 600, 605 in the treatment for mitral valve regurgitation. In the embodiments shown, the pledgets 600, 605 may be positioned against the annulus tissue to prevent tearing of the annulus tissue when the knot replacement tool 400 is pulling the first anchor 300 and the second anchor 310 towards each other, and when the knot replacement tool 400 is tying the first and second tethers 305, 315 together. The pledgets 600 605 are design to be delivered on the first and second tethers 305, 315 and contact the annulus tissue of the mitral valve at location C and location D.

The pledgets 600, 605 may be made from biocompatible flexible materials such as foam, felt or fabric. Pledgets may also be made from non-resorbable polyurethane, polyamide, polyethylene, polypropylene, polyethylene terephthalate, polytetrafluoroethylene (PTFE or Teflon®), or various absorbable polymers, such as polyglycolic acid or even pieces of autologous tissue. Pledgets may be formed in different shapes, such as circular or rectangular.

The treatment for mitral valve regurgitation includes systems and methods of modifying the abnormal or damaged mitral valve 180 to operate as a normal mitral valve to stop the mitral valve regurgitation. This is done by attaching first and second anchors in the annulus above the posterior leaflet 190 and pulling the first and second anchors toward each other to reshape the posterior leaflet 190. The pledgets are used to prevent tearing of the annulus tissue when tension is applied to move the first and second anchors toward each other. It may take multiple anchors that are pulled together to completely reshape the posterior leaflet.

FIG. 15A is a top view and 15B is a sectional view at F-F showing the first anchor 300 attached to the mitral valve annulus at location C and the second anchor 310 attached to the mitral valve annulus at location D. First and second pledgets 600, 605 are advanced along the first and second tethers 305, 315 toward locations C and D. Following delivery of the pledgets 600, 605, the knot replacement tool 400 is advanced along the first and second tethers 305, 315.

FIG. 16A is a top view and 16B is a sectional view at G-G showing the first pledget 600 positioned at location C and the second pledget 605 positioned at location D. The knot replacement tool 400 is then advanced along the first and second tethers 305, 315. While advancing, the knot replacement tool 400 applies tension to the first and second tethers 305, 315 so that the anchor 300 at location C and the anchor 310 at location D move toward each other.

Once the posterior leaflet 185 had been reshaped the desired amount, the knot replacement tool 400 ties the first and second tethers 305, 315, locking the reshaped posterior leaflet 190 in place.

A cutter 500 follows the first and second tethers 305, 315 to the knot replacement tool 400. The cutter 500 cuts the proximal end of the first and second tethers 505, 515 and then is withdrawn from the guide catheter 100.

FIG. 17A is a top view and 17B is a sectional view at H-H showing the knot replacement tool 400 in the locked position with the first and second pledgets 600, 605 positioned against the annulus tissue on the first and second tethers 305, 315, locking the posterior leaflet 190 in the desired shape, creating the modified mitral valve 180, with the anterior leaflet 185 and the posterior leaflet 190 functioning as a mitral valve.

Tricuspid Valve Repair

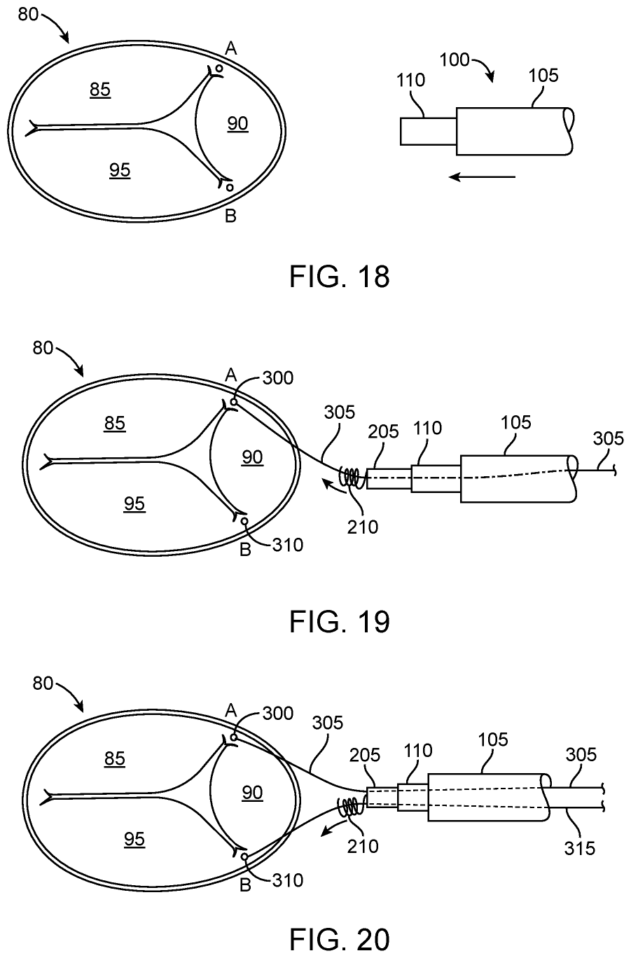

FIG. 18 shows a delivery system 100 having a guide catheter 105 that enters in the femoral vein and is advanced through the vascular system to the heart and into the right atrium 15. A second steerable sheath 110, such as an inner steerable/guide, is inserted through the guide catheter 105 and exits the distal end and advanced to the tricuspid valve 80.

FIG. 19 shows an inner steerable guide catheter 200 and an integrated therapy catheter 205 advanced through a lumen in the guide catheter 105 or the second steerable sheath 110. The integrated therapy catheter 205 is advanced to the tricuspid valve 80 and a valve engagement structure 210 extends from the distal end. See FIGs. ______ showing the steps used with the integrated therapy catheter 205 and valve engagement structure 210.

The valve engagement structure 210 engages the tricuspid valve 80 at junction A to hold the integrated therapy catheter 205 in place while a cannulated needle 220 punctures the tricuspid valve 80 in the tricuspid valve annulus at junction A of the anterior leaflet 85 and posterior leaflet 90. The integrated therapy catheter 205 then delivers a first anchor 300 having an attached tether 305 through junction A. The first anchor 300 expands or unfolds and engages the annulus of the tricuspid valve 80. The integrated therapy catheter 205 is then disengaged from the tricuspid valve 80.

In some embodiments, the valve engagement structure 210 is a spiral needle or corkscrew 210 configured to screw into the tricuspid valve annulus near junction A of the anterior leaflet 85 and posterior leaflet 90. The corkscrew 210 may be made from stainless steel or nitinol. The corkscrew 210 holds the integrated therapy catheter 205 in place as a cannulated needle 220 within the integrated therapy catheter 205 advances distally and punctures the tricuspid valve 80 at junction A of the anterior leaflet 85 and posterior leaflet 90.

FIG. 20 shows the integrated therapy catheter 205 advanced to the opposite side of the tricuspid valve 80, the corkscrew 210 on the distal end is screwed into the tricuspid valve 80 at junction B to hold the integrated therapy catheter 205 in place while a cannulated needle 220 punctures the tricuspid valve 80 in the tricuspid valve annulus at junction B of the posterior leaflet 90 and the septal leaflet 95. The integrated therapy catheter 205 then delivers a second anchor 310 having a second tether 315 through junction B in the tricuspid valve 80. The second anchor 310 is expanded or unfolds and engages the annulus of the tricuspid valve 80.

In some embodiments, the corkscrew 210 is screwed into the annulus near junction B of the posterior leaflet 90 and the septal leaflet 95. The corkscrew 210 holds the integrated therapy catheter 205 in place as a cannulated needle 220 within the integrated therapy catheter 205 advances distally and punctures the tricuspid valve 80 at junction B of the posterior leaflet 90 and the septal leaflet 95.

After the second anchor 310 is secured, the corkscrew 210 is unscrewed and the integrated therapy catheter 205 is withdrawn from the tricuspid valve 80, exposing the first and second tethers 305, 315 attached to the first and second anchors 300, 310.

The integrated therapy catheter 205 is then withdrawn from the lumen of the outer steerable catheter 100. After withdrawal, the proximal ends of first and second tethers 305, 315 extend from the proximal end of the lumen of the catheter.

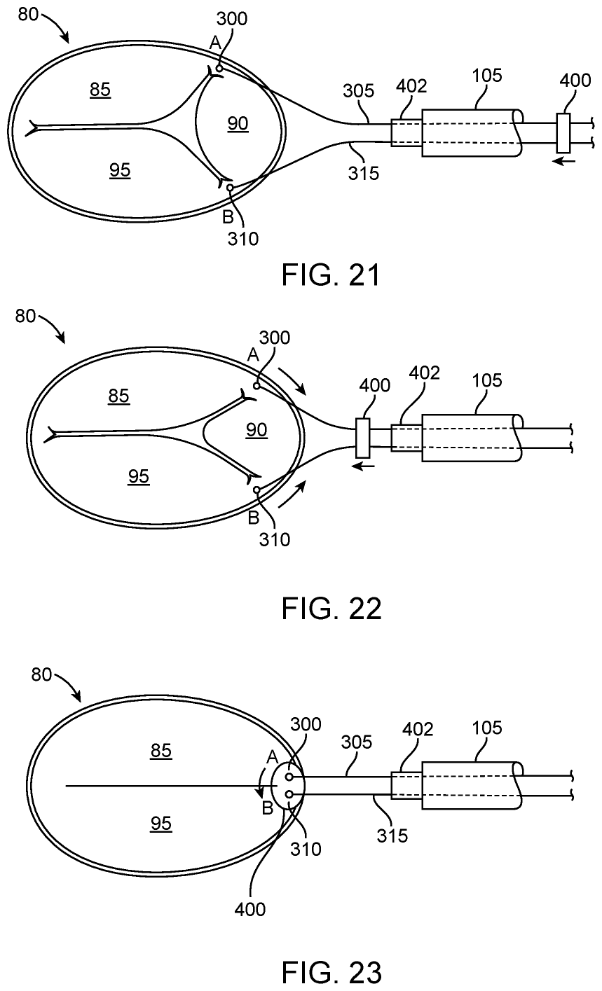

FIG. 21 shows a knot replacement delivery catheter 402 inserted into the lumen of the catheter 100. The proximal end of the first and second tethers 305, 315 are inserted into a knot replacement tool 400. The knot replacement tool 400 uses the first and second tethers 305, 315 as a rail to guide it to the tricuspid valve 80.

FIG. 22 shows the knot replacement tool 400 advancing over the first and second tethers 305, 315 toward the tricuspid valve 80. The advancing movement of the knot replacement tool 400 tensions the first and second tethers 305, 315 and pulls the first and second anchors 300, 310 toward each other, reducing the distance between junction A and junction B.

FIG. 23 shows the final position of the first and second anchors 300, 310 and the distance between junction A and junction B. In this position, the anterior leaflet 85 is next to the septal leaflet 95. The knot replacement tool 400 ties the first and second tethers 305, 315 together, locking them in place. The knot replacement delivery shaft 402 is then detached from the knot replacement tool 400 and withdrawn.

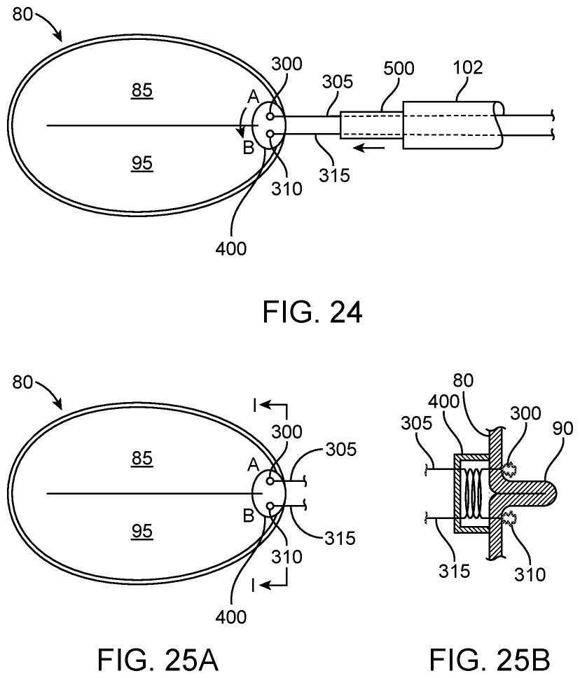

FIG. 24 shows the proximal ends of the first and second tethers 305, 315 inserted into a tether cutter 500. The tether cutter 500 follows the first and second tethers 305, 315 to the knot replacement tool 400. The tether cutter 500 then cuts the first and second tethers 305, 315. The tether cutter 500 is then withdrawn and finally the catheter 105 is withdrawn from the body.

FIG. 25A is a top view and 25B is a sectional view at I-I showing the knot replacement tool 400 in the locked position with the first and second tethers 305, 315, locking the anterior leaflet 85 and septal leaflet 95 in place creating the modified tricuspid valve 80, with the anterior leaflet 85 and the septal leaflet 95 functioning as a bicuspid valve.

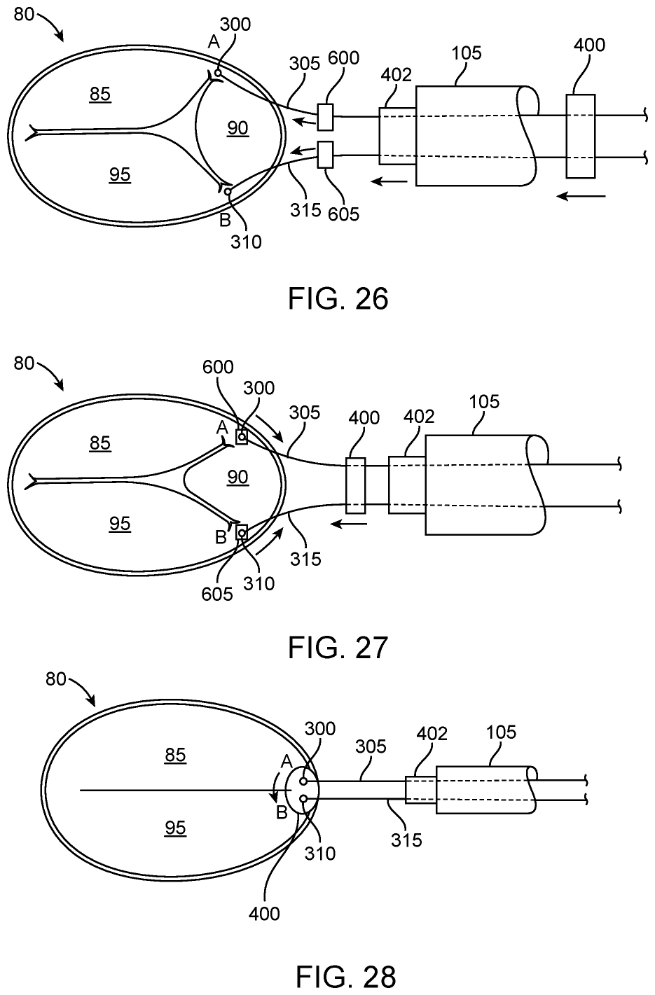

FIGS. 26-30B are similar to FIGS. 21-25B with the addition of first and second pledgets 600, 605 that are incorporated in the treatment for tricuspid valve regurgitation. The treatment includes systems and methods of modifying the abnormal or damaged tricuspid valve 80 to operate as a bicuspid valve to stop the tricuspid valve regurgitation. This is done by attaching first and second anchors in the annulus above the damaged leaflets. The first and second pledgets 600, 605 are used to prevent tearing of the anchors 300, 310 through the annulus tissue when tension is applied to move anchors 300, 310 toward each other to create the bicuspid valve using the anterior leaflet and septal leaflet. It may take multiple anchors that are pulled together to completely isolate the posterior leaflet.

FIG. 26 shows the first anchor 300 with the first tether 305 attached to the tricuspid valve annulus at junction A, and the second anchor 310 with second tether 315 attached to the tricuspid valve annulus at junction B. The first pledget 600 is slid over the proximal end of the first tether 305 and the second pledget 605 is slid over the proximal end of the second tether 315. The first and second pledgets 600, 605 are inserted through the lumen of the guide catheter 105 and advanced toward junctions A and B. The knot replacement delivery catheter 402 inserted into the lumen of the catheter 100. The proximal end of the first and second tethers 305, 315 are inserted into a knot replacement tool 400. The knot replacement tool 400 uses the first and second tethers 305, 315 as a rail to guide it to the tricuspid valve 80.

FIG. 27 shows the first pledget 600 positioned on the tricuspid valve annulus tissue at junction A, opposite the first anchor 300, and the second pledget 605 positioned on the annulus tissue at junction B, opposite the second anchor 310.

The knot replacement tool 400 is advance over the first and second tethers 305, 315 toward the tricuspid valve 80. The advancing movement of the knot replacement tool 400 tensions the first and second tethers 305, 315 and pulls the first and second anchors 300, 310 toward each other, reducing the distance between junction A and junction B. The first and second pledgets 600, 605 are used to provide support on the tricuspid valve annulus to prevent the first and second anchors 300, 310 from tearing through the tricuspid valve annulus tissue.

FIG. 28 shows the final position of the first and second anchors 300, 310 and the distance between junction A and junction B. In this position, the anterior leaflet 85 is next to the septal leaflet 95. The knot replacement tool 400 is rotated to tie the first and second tethers 305, 315 together, locking them in place. The knot replacement delivery shaft 402 is then detached from the knot replacement tool 400 and withdrawn.

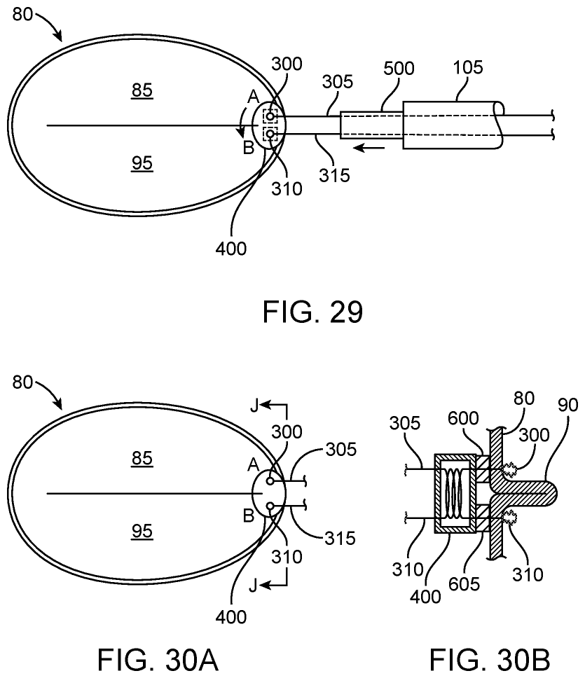

FIG. 29 shows a tether cutter 500 sliding along the first and second tethers 305, 315 through the lumen of the guide catheter 105 and following the first and second tethers 305, 315 to the knot replacement tool 400. The tether cutter 500 then cuts the first and second tethers 305, 315 near the knot replacement tool 400. The tether cutter 500 is then withdrawn and finally the catheter 105 is withdrawn from the body.

FIG. 30A is a top view and 30B is a sectional view at J-J showing the knot replacement tool 400 in the locked position with the first and second pledgets 600, 605 positioned against the tricuspid valve annulus tissue on the first and second tethers 305, 315, locking the anterior leaflet 85 and septal leaflet 95 in place, creating the modified tricuspid valve 80, with the anterior leaflet 85 and the septal leaflet 95 functioning as a bicuspid valve.

Mitral Valve Repair

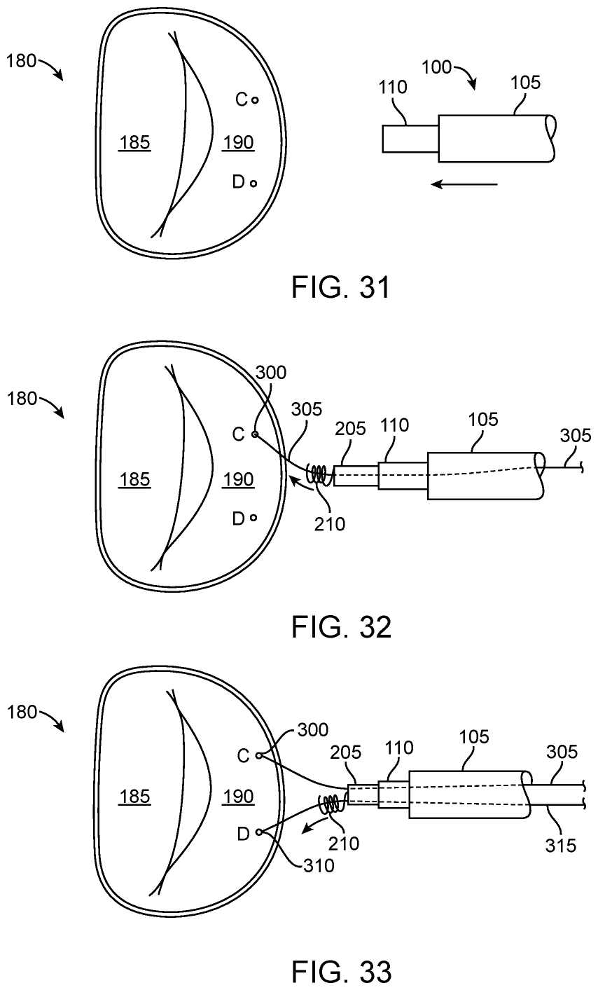

FIG. 31 shows a delivery system 100 having a guide catheter 105 that enters in the femoral vein and is advanced through the vascular system to the heart and into the right atrium 15. A second steerable sheath 110, such as an inner steerable/guide, is inserted through the guide catheter 105 and exits the distal end and advanced to the tricuspid valve 80.

FIG. 32 shows an inner steerable guide catheter 200 and an integrated therapy catheter 205 advanced through a lumen in the guide catheter 105 or the second steerable sheath 110. The integrated therapy catheter 205 is advanced to the mitral valve 180 and a valve engagement structure 210 extends from the distal end.

The valve engagement structure 210 engages the mitral valve 180 at location C to hold the integrated therapy catheter 205 in place while a cannulated needle 220 punctures the mitral valve 180 in the mitral valve annulus at location C of the anterior the posterior leaflet 190. The integrated therapy catheter 205 then delivers a first anchor 300 having an attached tether 305 through location C. The first anchor 300 expands or unfolds and engages the annulus of the mitral valve 180. The integrated therapy catheter 205 is then disengaged from the mitral valve 180.

In some embodiments, the valve engagement structure 210 is a spiral needle or corkscrew 210 configured to screw into the mitral valve annulus near location C of the posterior leaflet 190. The corkscrew 210 may be made from stainless steel or nitinol. The corkscrew 210 holds the integrated therapy catheter 205 in place as a cannulated needle 220 within the integrated therapy catheter 205 advances distally and punctures the mitral valve 180 at location C of the posterior leaflet 90.

FIG. 33 shows the integrated therapy catheter 205 advanced to location D of the mitral valve 80, the corkscrew 210 on the distal end is screwed into the mitral valve 180 at location D to hold the integrated therapy catheter 205 in place while a cannulated needle 220 punctures the mitral valve 180 in the mitral valve annulus at location D of the posterior leaflet 190. The integrated therapy catheter 205 then delivers a second anchor 310 having a second tether 315 through location D in the mitral valve 180. The second anchor 310 is expanded or unfolds and engages the annulus of the mitral valve 180.

In some embodiments, the corkscrew 210 is screwed into the annulus near location D of the posterior leaflet 90. The corkscrew 210 holds the integrated therapy catheter 205 in place as a cannulated needle 220 within the integrated therapy catheter 205 advances distally and punctures the mitral valve 180 at location D of the posterior leaflet 90.

After the second anchor 310 is secured, the corkscrew 210 is unscrewed and the integrated therapy catheter 205 is withdrawn from the mitral valve 180, exposing the first and second tethers 305, 315 attached to the first and second anchors 300, 310.

The integrated therapy catheter 205 is then withdrawn from the lumen of the outer steerable catheter 100. After withdrawal, the proximal ends of first and second tethers 305, 315 extend from the proximal end of the lumen of the catheter.

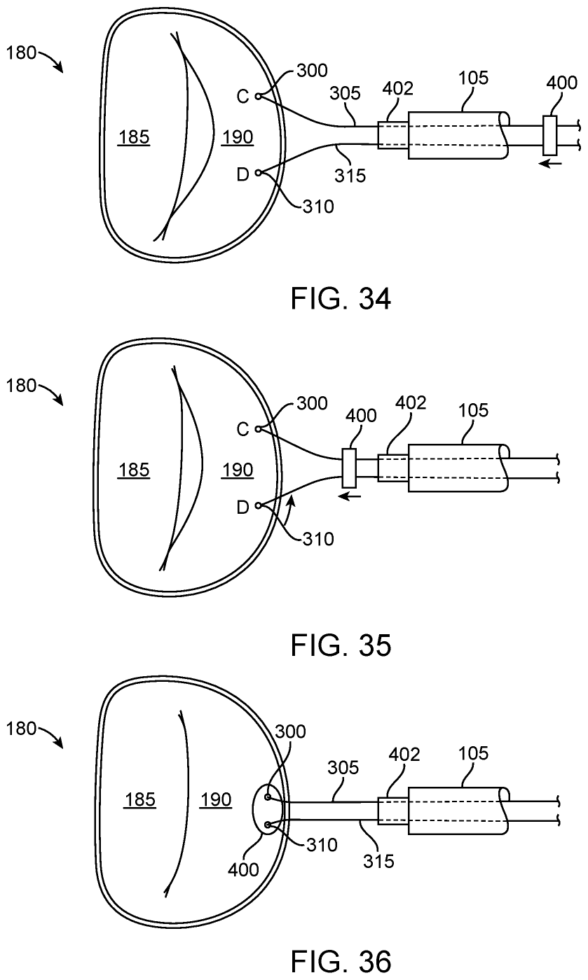

FIG. 34 shows a knot replacement delivery catheter 402 inserted into the lumen of the catheter 100. The proximal end of the first and second tethers 305, 315 are inserted into a knot replacement tool 400. The knot replacement tool 400 uses the first and second tethers 305, 315 as a rail to guide it to the tricuspid valve 80.

FIG. 34 shows the knot replacement tool 400 advancing over the first and second tethers 305, 315 toward the mitral valve 180. The advancing movement of the knot replacement tool 400 tensions the first and second tethers 305, 315 and pulls the first and second anchors 300, 310 toward each other, reducing the distance between location C and location D.

FIG. 36 shows the final position of the first and second anchors 300, 310 and the distance between location C and location D. In this position, the posterior leaflet 190 has been reshaped. The knot replacement tool 400 ties the first and second tethers 305, 315 together, locking them in place. The knot replacement delivery shaft 402 is then detached from the knot replacement tool 400 and withdrawn.

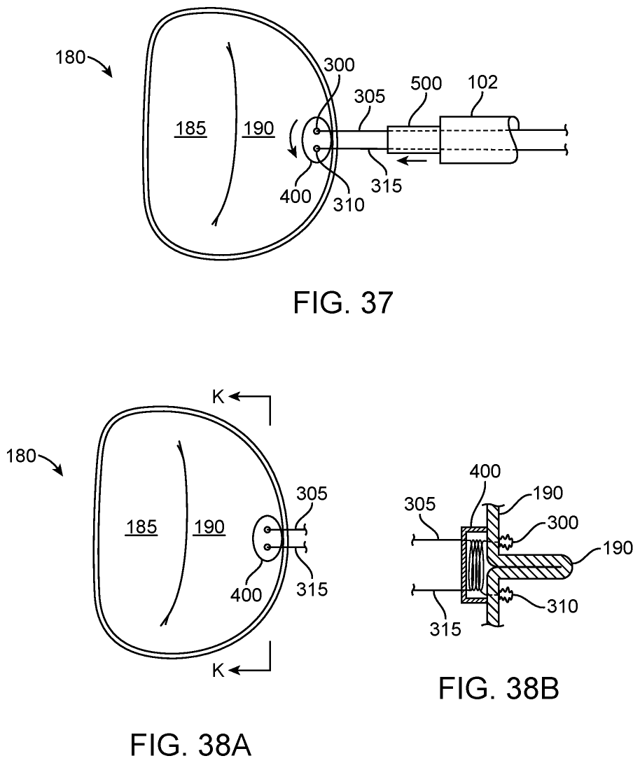

FIG. 37 shows the proximal ends of the first and second tethers 305, 315 inserted into a tether cutter 500. The tether cutter 500 follows the first and second tethers 305, 315 to the knot replacement tool 400. The tether cutter 500 then cuts the first and second tethers 305, 315. The tether cutter 500 is then withdrawn and finally the catheter 105 is withdrawn from the body.

FIG. 38A is a top view and 38B is a sectional view at K-K showing the knot replacement tool 400 in the locked position with the first and second tethers 305, 315, locking the posterior leaflet 190 in place creating the modified mitral valve 180, with the anterior leaflet 185 and the posterior leaflet 190 functioning as a normal mitral valve.

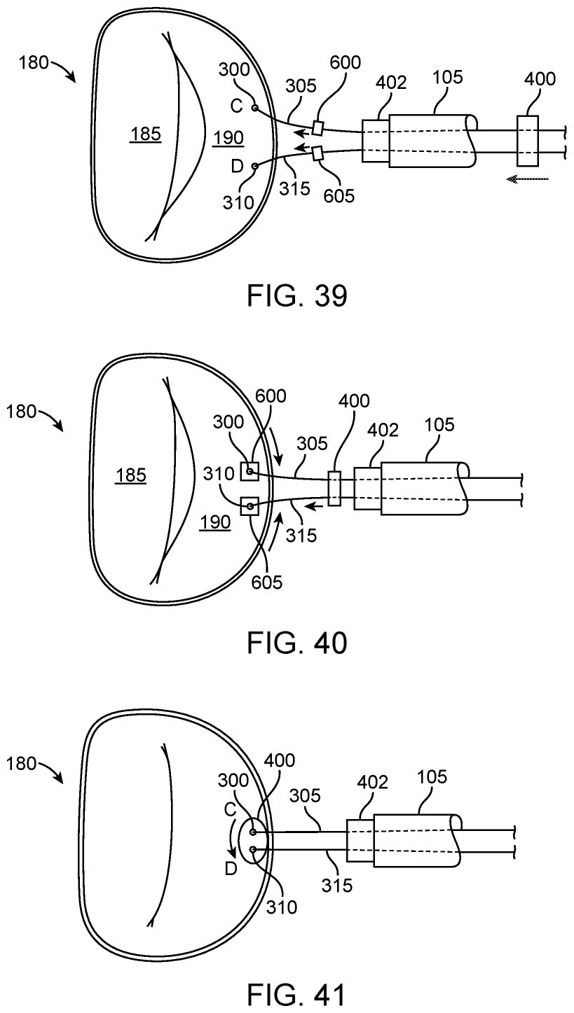

FIGS. 39-43B are similar to FIGS. 34-38B with the addition of first and second pledgets 600, 605 that are incorporated in the treatment for mitral valve regurgitation. The treatment includes systems and methods of modifying the abnormal or damaged mitral valve 180 to operate as a normal mitral valve to stop the mitral valve regurgitation. This is done by attaching first and second anchors in the annulus with first and second pledgets 600, 605 to prevent tearing of the anchors 300, 310 through the annulus tissue when tension is applied to move anchors 300, 310 toward each other. It may take multiple anchors that are pulled together to completely reshape the posterior leaflet 190.

FIG. 39 shows the first anchor 300 with the first tether 305 attached to the mitral valve annulus at location C, and the second anchor 310 with second tether 315 attached to the mitral valve annulus at location D. The first pledget 600 is slid over the proximal end of the first tether 305 and the second pledget 605 is slid over the proximal end of the second tether 315. The first and second pledgets 600, 605 are inserted through the lumen of the guide catheter 105 and advanced toward locations C and D. The knot replacement delivery catheter 402 inserted into the lumen of the catheter 100. The proximal end of the first and second tethers 305, 315 are inserted into a knot replacement tool 400. The knot replacement tool 400 uses the first and second tethers 305, 315 as a rail to guide it to the mitral valve 180.

FIG. 40 shows the first pledget 600 positioned on the mitral valve annulus tissue at location C, opposite the first anchor 300, and the second pledget 605 positioned on the mitral valve annulus tissue at location D, opposite the second anchor 310.

The knot replacement tool 400 is advance over the first and second tethers 305, 315 toward the mitral valve 180. The advancing movement of the knot replacement tool 400 tensions the first and second tethers 305, 315 and pulls the first and second anchors 300, 310 toward each other, reducing the distance between location C and location D. The first and second pledgets 600, 605 are used to provide support on the mitral valve annulus to prevent the first and second anchors 300, 310 from tearing through the mitral valve annulus tissue.

FIG. 41 shows the final position of the first and second anchors 300, 310 and the distance between location C and location D. In this position, the posterior leaflet 190 has been reshaped. The knot replacement tool 400 is rotated to tie the first and second tethers 305, 315 together, locking them in place. The knot replacement delivery shaft 402 is then detached from the knot replacement tool 400 and withdrawn.

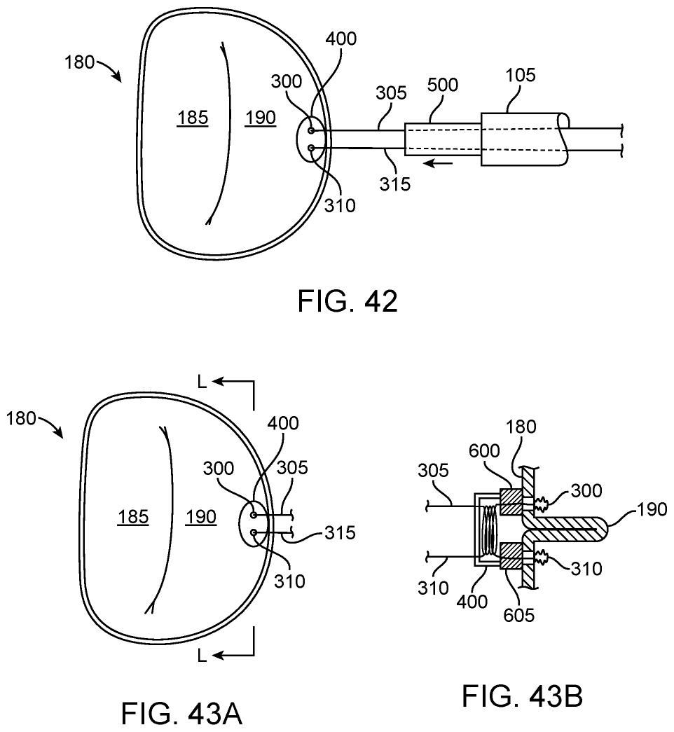

FIG. 42 shows a tether cutter 500 sliding along the first and second tethers 305, 315 through the lumen of the guide catheter 105 and following the first and second tethers 305, 315 to the knot replacement tool 400. The tether cutter 500 then cuts the first and second tethers 305, 315 near the knot replacement tool 400. The tether cutter 500 is then withdrawn and finally the catheter 105 is withdrawn from the body.

FIG. 43A is a top view and 43B is a sectional view at L-L showing the knot replacement tool 400 in the locked position with the first and second pledgets 600, 605 positioned against the mitral valve annulus tissue on the first and second tethers 305, 315, locking the posterior leaflet 190 in place, creating the modified mitral valve 180.

Integrated Therapy Catheter

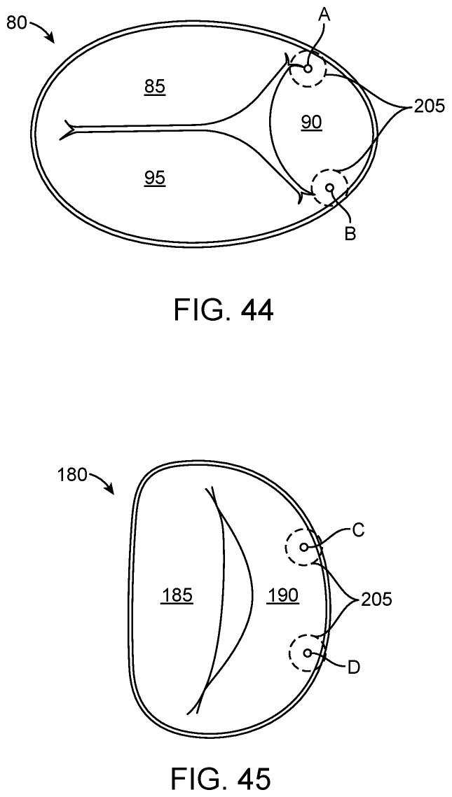

FIGS. 44-50 show various views of an integrated therapy catheter 205 configured to: engage locations A, B of a tricuspid valve 80 or locations C, D of a mitral valve 180 through active deflection of the integrated steerable sheath, puncture the locations, and deliver anchors into the locations (FIGS. 44, 45).

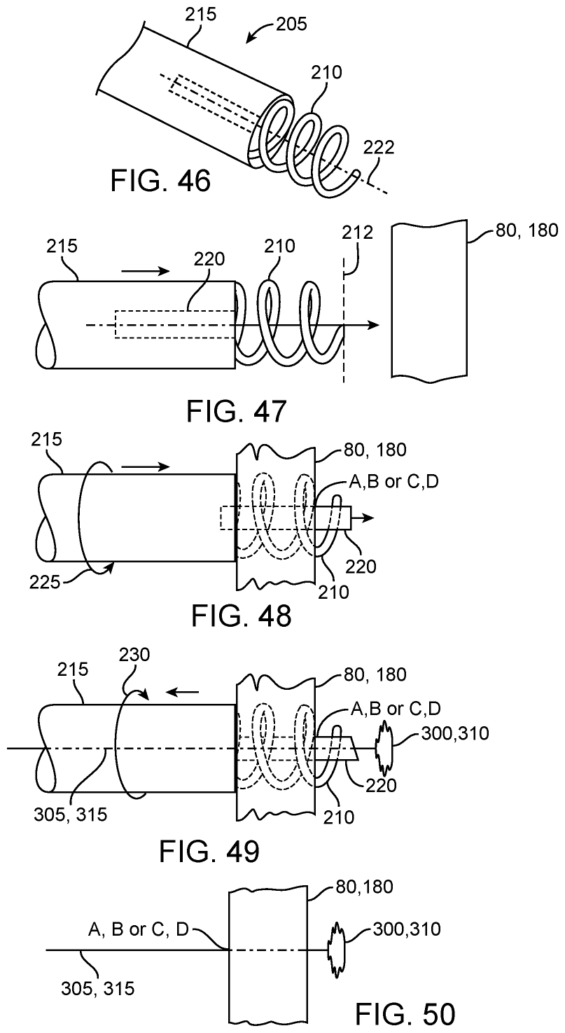

The integrated therapy catheter 205 includes a tissue engagement portion 210 coupled to the distal end of the body portion 215 (FIG. 46). The tissue engagement portion 210 is configured to rotate clockwise and engage the tricuspid valve 80 or mitral valve 180 and hold the integrated therapy catheter 205 while the needle 220 penetrates junctions A, B of the tricuspid valve annulus, or locations C, D of the mitral valve annulus (FIGS. 47, 48). The anchors 300, 305 are then delivered through the cannulated needle 220 (FIG. 49). The cannulated needle 220 is then withdrawn and the corkscrew 210 is rotated in a counterclockwise direction 230 to release the integrated therapy catheter 205 from the valve annulus and remove. The first and second anchors 300, 310 and first and second tethers 305, 315 stay attached to the tricuspid or mitral valve annulus (FIG. 50).

Knot Replacement Tool

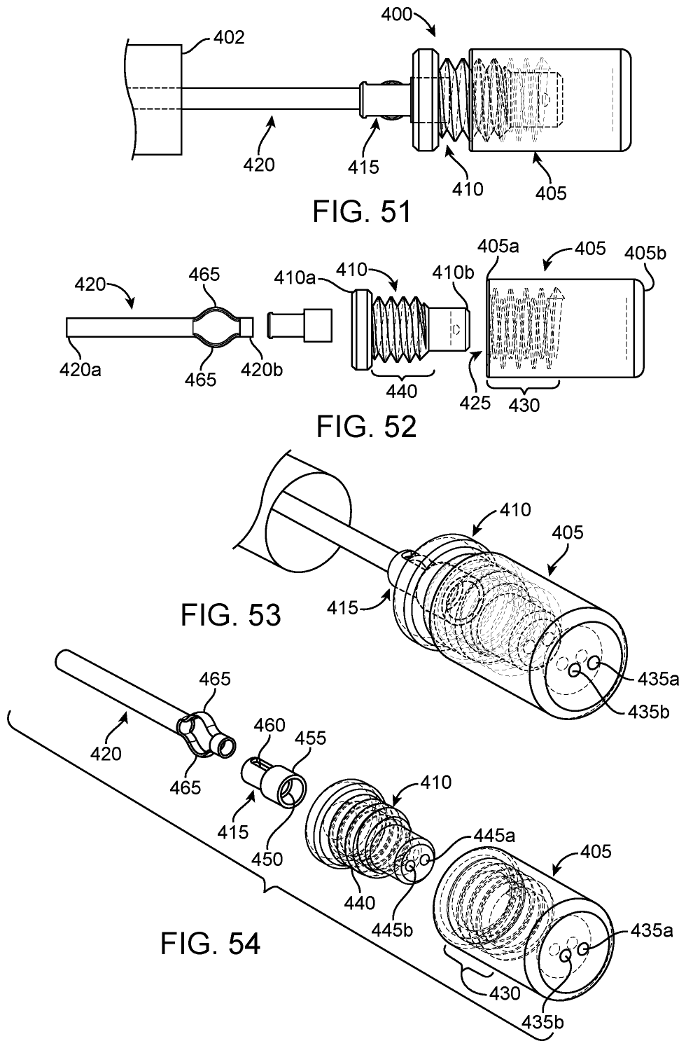

FIGS. 51-56 are views showing one embodiment of a knot replacement tool 400 that is configured to couple with the proximal end of the first and second tethers 305, 315 outside of the body. Once coupled, the first and second tethers 305, 315 are used as a rail to guide the knot replacement tool 400 to the targeted location at junctions A and B of the tricuspid valve 80 or locations C and D of the mitral valve 180.

FIG. 51 is a side view and FIG. 52 is a side exploded view of the knot replacement tool 400. FIG. 53 is a perspective view and FIG. 54 is a perspective exploded view of the knot replacement tool 400.

The knot replacement tool 400 includes a locking cap 405, a locking screw 410, a shaft coupler 415 and a delivery shaft 420.

The locking cap 405 is cylindrical in shape with a cylindrical cavity 425 open on a proximal end 405a and closed on a distal end 405b. The cylindrical cavity 425 includes an internal threaded portion 430 near the proximal end 405a and the closed end includes first and second tether locking cap holes, 435a, 435b sized to receive the first and second tethers 305, 315.

The locking screw 410 includes an external threaded portion 440 and a cylindrical cavity that is open on a proximal end 410a and closed on a distal end 410b. The closed end 410b includes first and second tether locking screw holes 445a, 445b sized to receive the first and second tethers 305, 315. The distal end 410b is configured to be inserted into the open proximal end 405a of the locking cap 405, and the external threaded portion 440 are configured to rotatingly engage with the internal threads 430 so that the locking screw 410 may be screwed into the locking cap 405.

During use, the first and second tethers 305, 315 are inserted through both the first and second tether locking cap holes 435a, 435b and then through the first and second tether locking screw holes 445a, 445b. As the locking screw 410 is rotated into the locking cap 405, the first and second tether locking screw holes, 445a, 445b are rotated with respect to the first and second tether locking cap holes, 435a, 435b. During this rotation, the first tether 315 and second tether 315 are twisted around each other and are locked in place.

Connect/Disconnect Feature

The shaft coupler 415 and a delivery shaft 420 are components of a connect/disconnect feature of the knot replacement tool 400 that are configured to couple the shaft 415 with the locking screw 410. The coupling of the shaft 420 allows torque to be applied to the locking screw 410. The design also has zero release force when the shaft 420 is uncoupled from the shaft coupler 415. This provides the ability to torque the locking screw 410 and screw the locking screw 410 into the locking cap 405. The shaft 420 can then be uncoupled without disrupting the locking screw 410 and the locking cap 405 when they are locked together.

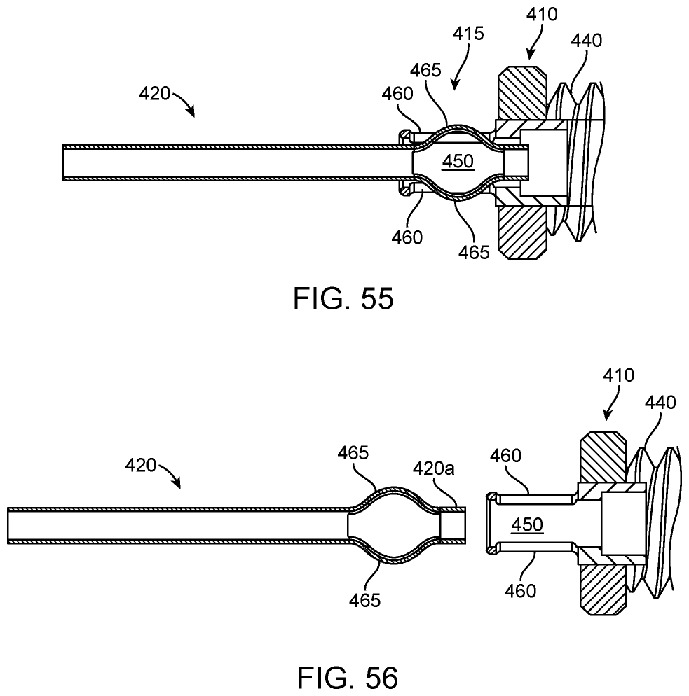

FIG. 55 is a sectional view showing the delivery shaft 420 coupled to the shaft coupler 415. The shaft coupler 415 is cylindrical in shape with a central opening 450 having a proximal portion 455 configured to be inserted into the cylindrical cavity 425 of the locking screw 410 and fixed in place. The shaft coupler 415 also includes slots 460.

The delivery shaft 420 includes a distal end 420a with engagement arms 465 having springlike properties that allow them to deflect and spring back to their original position. The distal end 420a is configured to be inserted into the into the central opening 450 of the shaft coupler 415.

The distal end of the delivery shaft 420 is sized for insertion into the central opening 450. During insertion, a curved distal portion of the engagement arms 465 contacts the shaft coupler 415 and deflects inwardly into the central opening 450 until the engagement arms 465 line up with the slots 460. Then the spring arms 465 return to their original shape and engage and lock in the slots 460. When the engagement arms 465 are coupled with the slots 460, the locking screw 410 may be rotated or torqued in the locking cap 405.

FIG. 56 is a sectional view showing the delivery shaft 420 disengaged from the shaft coupler 415. To disconnect the delivery shaft 420 from the shaft coupler 415, a removal tube is distally slid over the delivery shaft 420 until it engages the proximal end of the shaft coupler 415. Once the removal tool is engaged, the delivery shaft 420 is pulled proximally and the engagement arms 465 deflect inward, allowing removal of the delivery shaft 420 from the central opening of the shaft coupler 415.

Cutter

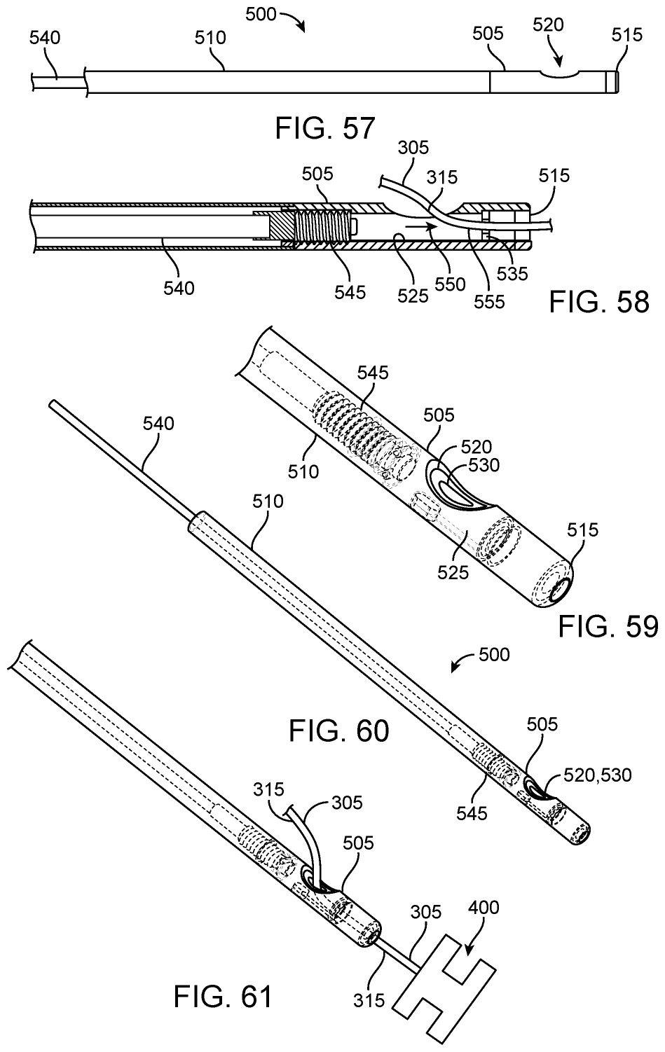

FIGS. 57-61 are views showing one embodiment of a cutter 500 that is configured to couple with the first and second tethers 305, 315 outside the body. The first and second tethers 305, 315 enter an opening 515 in the distal end and exit from inner and outer side openings 520, 530. The cutter 500 then uses the first and second tethers 305, 315 as a rail to guide the cutter 500 to the to the targeted location proximate the knot replacement tool 400 at the first and second leaflets 45a, 45b.

FIG. 57 is a side view and FIG. 58 is a sectional view of a cutter 500, FIG. 59 is a distal perspective view showing more details of the cutting area of the cutter 500, FIG. 60 is a perspective view of the cutter 500, and FIG. 61 is a distal perspective view showing routing of the first and second tethers 305, 315 through the cutter 500.

The cutter 500 includes an outer cutter body 505 coupled to a braided polyimide shaft 510 and a distal stopper with opening 515. The outer cutter body is a cylindrical tube with a side opening 520. An inner cutter body 525 is positioned within the outer cutter body 505. The inner cutter body 525 is a cylindrical tube with a side opening 530. The side opening 520 of the outer cutter body 505 and side opening 530 the inner cutter body 525 are positioned to provide an opening to a center lumen 555. A stopper 535 is coupled to the distal end of the inner cutter body 525 and include an opening to direct the first and second tethers 305, 315 through the outer and inner cutter bodies aligned side openings 520, 530. A high torque flexible cable or torque shaft 540 is slidably positioned within the lumen of the components. The high torque cable 540 is used to rotate a thread 545 that drives the inner cutter body 525 in an axial direction 550 to scissor cut the first and second tethers 305, 315 between the sharp edges of the inner cutter opening 530 and outer cutter opening 520. The distal end of the flexible cable 540 is positioned proximally of the side openings 520 and 530 during delivery. Once the cutter 500 is in the desired position, the first and second tethers 305, 315 are tensioned, and the flexible cable 540 is rotated to drive the threaded cutter mechanism that moves the inner cutter 525 relative to the outer cutter 505 to cut the first and second tethers 305, 315.

Using the Knot Replacement Tool

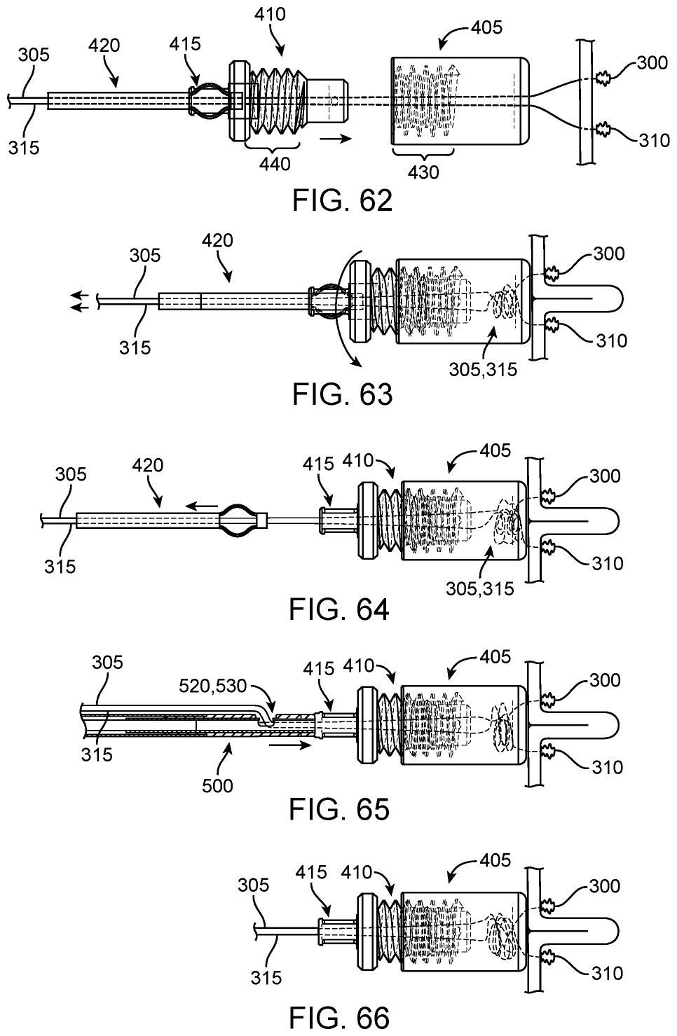

FIGS. 62-66 are views showing the knot replacement tool 400 engaging the first and second tethers 305, 315 attached to a valve annulus at positions A, B or positions C, D.

FIG. 62 shows the first and second tethers 305, 315 inserted through the first and second tether locking cap holes, the first and second locking screw holes, through the central opening in the shaft connector and the interior lumen of the deliver shaft. The knot replacement tool 400 uses the first and second tethers 305, 315 as a rail to guide the knot replacement tool 400 to the targeted location.

FIG. 63 shows tensioning of the first and second tethers 305, 315 to pull the first and second junctions together to engage the locking cap 405. The locking screw 410 is then inserted into the locking cap 405 and the delivery shaft 420 rotates the locking screw 410. At the same time, the first tether 305 and second tether 315 are twisted around each other and locked in place.

FIG. 64 shows the delivery shaft 420 being disconnected from the shaft coupler 415 and removed.

FIG. 65 shows a cutter 500 using the first and second tethers 305, 315 as a rail to the targeted location proximate the knot replacement tool 400. The cutter 500 cuts the first and second tethers 305, 315 on the proximal side of the shaft coupler 415. The cutter 500 is then removed along with the cut portions of the first and second tethers 305, 315.

FIG. 66 shows the modified valve with the junctions next to each other.

While the embodiments above show treatment of the tricuspid or mitral valve attaching first and second anchors at first and second locations, more than two anchors may be used (multiple anchors) at more than two locations (multiple locations).

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A system to repair a tricuspid or mitral valve comprising:
an integrated therapy catheter configured to engage the annulus of the tricuspid or mitral valve at a first location and a second location having:
a valve engagement structure at a distal end configured to couple with the annulus at the first location and the second location;
a puncture device configured to puncture a first hole in the annulus at the first location and a second hole in the annulus at the second location; and
a delivery device configured to deliver a first anchor through the first hole and a second anchor through the second hole;
the first anchor having a self-expanding distal end and a proximal tether configured to be delivered through the first hole by the delivery device, the self-expanding distal end configured to expand larger than the first hole and engage the annulus of the tricuspid or mitral valve;
a second anchor having a self-expanding distal end and a proximal tether configured to be delivered through the second hole by the delivery device, the self-expanding distal end configured to expand larger than the second hole and engage the annulus of the tricuspid or mitral valve; and
a knot replacement tool configured to couple with the first and second tethers, tension the first and second tethers to bring the first location and the second location toward each other, and lock the first and second tethers.

2. The system of claim 1, wherein the valve engagement structure includes a spiral needle or corkscrew coupled to a distal end of a body portion, wherein rotation of the spiral needle or corkscrew in a first direction screws the spiral needle or corkscrew into the annulus of the tricuspid or mitral valve.

3. The system of claim 2, wherein rotation of the spiral needle or corkscrew in a second direction unscrews the spiral needle or corkscrew from the annulus of the tricuspid or mitral valve.

4. The system of claim 1, wherein the puncture device includes a cannulated needle positioned within the proximal body configured to extend distally to puncture the first and second holes in the annulus at the first location and the second location.

5. The system of claim 4, wherein delivery device includes delivery of the first and second anchors through the cannulated needle to the first and second holes.

6. The system of claim 1, wherein the knot replacement tool includes:
a locking cap is cylindrical body in shape with an internal threaded portion and a closed distal end having first and second tether locking cap holes; and
a locking screw is cylindrical body in shape with an external threaded portion and a closed distal end having first and second tether locking screw holes;
wherein the locking screw is configured to be inserted into the locking cap and the external threaded portion configured to rotatingly engage with the internal threaded portion so that the locking screw may be screwed into the locking cap.

7. The system of claim 6, wherein as the locking screw is rotated in the locking cap, the first and second tether locking screw holes and the first and second tether locking cap holes are rotated in opposite directions and the first tether and second tether are twisted around each other and are locked in place.

8. The system of claim 6, wherein knot replacement tool further includes a detachable delivery shaft coupled with the locking screw.

9. The system of claim 1, further comprising a cutter device configured to cut the first and second tethers.

10. The system of claim 9, wherein cutter device includes:
an outer body that is a cylindrical tube with outer body side opening; and
an inner body slidably positioned within the outer body, the inner body is a cylindrical tube with an inner body side opening, the inner body side opening and the outer body side opening are positioned to provide an opening to a center lumen,
wherein the first and second tethers enter the distal end of the center lumen and exit out of the center lumen through the inner body side opening and outer body side opening, and
axial movement of the inner body scissor cuts the first and second tethers between the edges of the inner body side opening and outer body side opening.

11. A system to repair a tricuspid or mitral valve comprising:
an integrated therapy catheter configured to puncture first and second holes in the valve annulus of the tricuspid or mitral valve at first and second locations;
an anchor delivery device configured to deliver first and second anchors to the first and second holes, the first and second anchors having first and second tethers with distal ends configured couple with the first and second holes; and
a knot replacement tool configured to couple with the first and second tethers to move the first and second locations toward each other a desired distance and lock the first and second tethers at the desired distance to modify one or more valve leaflets.

12. The system of claim 11, wherein the distal end of the first and second anchors are configured to be delivered through the first and second holes by the delivery device and self-expand to engage the annulus.

13. The system of claim 11, wherein the tricuspid valve includes the first location at a junction above the anterior leaflet and posterior leaflet, and the second location at a junction above the posterior leaflet and septal leaflet.

14. The system of claim 11, wherein the mitral valve includes first and second locations above the posterior leaflet.

15. The system of claim 11, wherein the integrated therapy catheter includes:
a valve engagement structure configured to couple with the valve annulus; and
a puncture device configured to puncture the first and second holes in the valve annulus.

16. The system of claim 15, wherein the valve engagement structure includes a spiral needle or corkscrew coupled to a distal end of a body portion, wherein rotation of the spiral needle or corkscrew in a first direction screws the spiral needle or corkscrew into the valve annulus and rotation of the spiral needle or corkscrew in a second direction unscrews the spiral needle or corkscrew from the valve annulus.

17. The system of claim 11, wherein the knot replacement tool includes:
a locking cap having first and second tether locking cap holes; and
a locking screw having first and second tether locking screw holes;
wherein the locking screw is configured to be screwed into the locking cap and the first and second tether locking cap holes and first and second tether locking screw holes are rotated in opposite directions and the first tether and second tether are twisted around each other and are locked in place.

18. The system of claim 11, further comprising a cutter device configured to cut the first and second tethers.

19. A method for repairing a tricuspid or mitral valve comprising:
puncturing first and second holes in an annulus of the tricuspid or mitral valve at first and second locations;
delivering first and second anchors to the first and second holes;
coupling the first and second anchors with the first and second holes;
moving the first and second holes toward each other using a knot replacement tool configured to tension the first and second tethers; and
locking the first and second tethers using a knot replacement tool configured to couple with the first and second tethers.

20. The method of claim 19, wherein the valve engagement structure includes a spiral needle or corkscrew coupled to a distal end of a body portion, wherein rotation of the spiral needle or corkscrew in a first direction screws the spiral needle or corkscrew into the valve annulus and rotation of the spiral needle or corkscrew in a second direction unscrews the spiral needle or corkscrew from the valve annulus.
